(19) United States
(12) Patent Application Publication
(10) Pub. No.: US 2022/0095929 A1
(43) Pub. Date: Mar. 31, 2022

(54) BIOMEDICAL SIGNAL SENSING DEVICE AND METHOD

(71) Applicant: AU Optronics Corporation, Hsin-chu (TW)

(72) Inventors: Yi-Han CHANG, HSIN-CHU (TW); Chung-Chin HUANG, HSIN-CHU (TW); Ching-Hua HSU, HSIN-CHU (TW); Min-Qian JIANG, HSIN-CHU (TW); Hao-Lun HSIEH, HSIN-CHU (TW)

(21) Appl. No.: 17/225,498

(22) Filed: Apr. 8, 2021

(30) Foreign Application Priority Data
Sep. 26, 2020 (TW) 109133536

(51) Int. Cl. A61B 5/021; A61B 5/00

(52) U.S. Cl. CPC A61B 5/021; A61B 2562/0247; A61B 5/6824

(57) ABSTRACT

A biomedical signal sensing device includes a frame, a first carrier, and a sensing module. The frame includes a base, a first adjusting arm, and a second adjusting arm connecting to the base through the first adjusting arm. The first carrier is disposed on the base and includes first and second grooves. The sensing module is connected to the first adjusting arm through the second adjusting arm. The sensing module includes a second carrier connected to the second adjusting arm, and a pressure sensor disposed on the second carrier, in which the pressure sensor faces towards the first groove or the second groove. The first groove and the second groove extend along a first direction, the second adjusting arm extends along a second direction, the first adjusting arm extends along a third direction, and the first direction, the second direction, and the third direction are perpendicular to each other.

### RELATED APPLICATIONS

This application claims priority to Taiwanese Application Serial Number 109133536, filed Sep. 26, 2020, which is herein incorporated by reference.

### BACKGROUND

### Field of Invention

The present invention relates to a sensing device and method. More particularly, the present invention relates to a biomedical signal sensing device and method.

### Description of Related Art

Recent signal processing techniques can analyze user's health information by biomedical signals from the user. In current biomedical signals, pulse is one of main signals for understanding the health information in the field of traditional Chinese medicine.

A pulse analysis apparatus can be utilized to record the wave of the pulse that traditional Chinese medicine requires. However, the pulse analysis apparatus fails to provide a precise positioning method and thus the user is unable to rest or be pressed at a correct position. The accuracy and the analysis result of the pulse analysis apparatus are unsatisfied. Furthermore, the pulse analysis apparatus cannot fit both left-hand and right-hand modes. Based on above reasons, a detecting device having a better position mechanism and accuracy is required.

### SUMMARY

According to some embodiments of the invention, a biomedical signal sensing device includes a frame, a first carrier, and a sensing module. The frame includes a base, a first adjusting arm, and a second adjusting arm connecting to the base through the first adjusting arm. The first carrier is disposed on the base and includes a first groove and a second groove disposed adjacent the first groove. The sensing module is connected to the first adjusting arm through the second adjusting arm. The sensing module includes a second carrier connected to the second adjusting arm, and a pressure sensor disposed on the second carrier, in which the pressure sensor faces towards the first groove or the second groove. The first groove and the second groove extend along a first direction, the second adjusting arm extends along a second direction, the first adjusting arm extends along a third direction, and the first direction, the second direction, and the third direction are perpendicular to each other.

According to some embodiments of the invention, a biomedical signal sensing method for taking a biomedical signal from a user includes providing a biomedical signal sensing device, which comprises a frame, a first carrier, and a sensing module. A wrist of the user is rested on the first carrier of the biomedical signal sensing device, and a position of the wrist relative to the first carrier on a first direction is determined. A position of the sensing module on a second direction is adjusted by the frame. The position of the sensing module on a third direction is adjusted by the frame, and the sensing module is forced touching the wrist. The biomedical signal is read by the sensing module.

It is to be understood that both the foregoing general description and the following detailed description are by examples, and are intended to provide further explanation of the invention as claimed.

### BRIEF DESCRIPTION OF THE DRAWINGS

The accompanying drawings are included to provide a further understanding of the invention, and are incorporated in and constitute a part of this specification. The drawings illustrate embodiments of the invention and, together with the description, serve to explain the principles of the invention. In the drawings,

FIG. 1 is an oblique view of a biomedical signal sensing device, according to some embodiments of the invention;

FIG. 2 is a schematic top view of the biomedical signal sensing device, according to some embodiments of the invention;

FIG. 3 and FIG. 4 are schematic top views of the biomedical signal sensing device at different operation states, according to some embodiments of the invention;

FIG. 5 is an oblique view of the first carrier, according to some embodiments of invention; and

FIG. 6 and FIG. 7 are schematic side views of the biomedical signal sensing device at different operation stages, according to some embodiments of the invention.

### DESCRIPTION OF THE EMBODIMENTS

Reference will now be made in detail to the present embodiments of the invention, examples of which are illustrated in the accompanying drawings. Wherever possible, the same reference numbers are used in the drawings and the description to refer to the same or like parts.

The present invention provides a biomedical signal sensing device and a biomedical signal sensing method, which can be utilized in a pulse analysis apparatus or other apparatus that detecting human biomedical signal. The biomedical signal for example can be pulse, but the invention is not limited to.

It will be understood that, although the terms first, second, third etc. may be used herein to describe various elements, components, regions, layers and/or sections, these elements, components, regions, layers and/or sections should not be limited by these terms. These terms are only used to distinguish one element, component, region, layer or section from another element, component, region, layer or section. Thus, a first element, component, region, layer or section discussed below could be termed a second element, component, region, layer or section without departing from the teachings of the present invention.

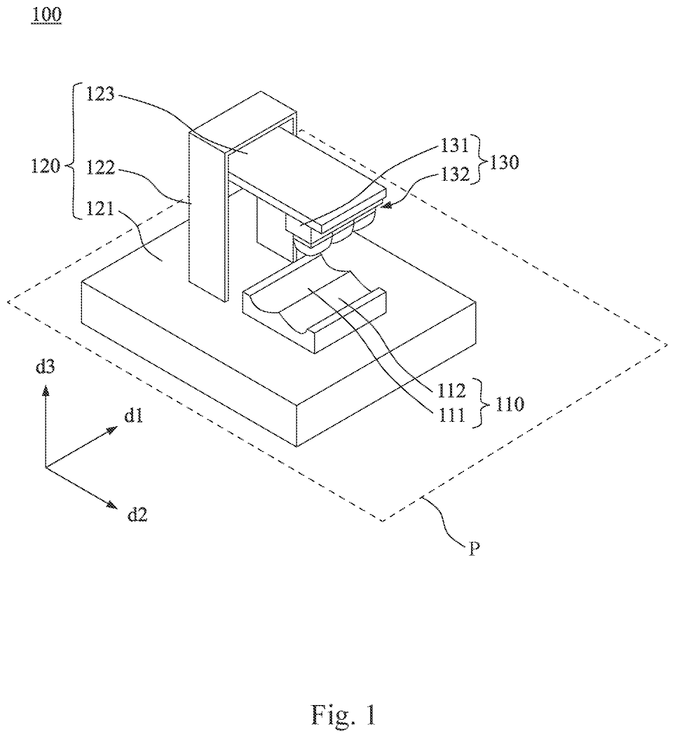

FIG. 1 is an oblique view of a biomedical signal sensing device, according to some embodiments of the invention. Please referring to FIG. 1, in some embodiments, the biomedical signal sensing device 100 includes a first carrier 110, a frame 120, and a sensing module 130. The frame 120 connects to the sensing module 130, and the first carrier 110 is disposed on the frame 120. Namely, the frame 120 connects the sensing module 130 to the first carrier 110.

For example, the frame 120 includes a base 121, a first adjusting arm 122, and a second adjusting arm 123. The base 121 is configured to be position on a firm horizontal plane, such as on a floor, a table, or the like, and the invention is not limited to. The first adjusting arm 122 connects the base 121 to the second adjusting arm 123. The first adjusting arm 122 extends along a third direction d3, and the second adjusting arm 123 extends along a second direction d2. The first adjusting arm 122 is configured to adjust a height of the second adjusting arm 123 along the third direction d3.

The first carrier 110 is disposed on the base 121 of the frame 120. The first carrier 110 includes a first groove 111 and a second groove 112, and the first groove 111 is disposed adjacent the second groove 112. The first groove 111 and the second groove 112 are both extend along the first direction d1, such that the wrist of the user is placed along the first direction d1.

More particularly, in some embodiments, when the base 121 of the frame 120 is placed on a plane P, the third direction d3 is substantially perpendicular to the plane P. The first direction d1 and the second direction d2 are perpendicular to the third direction d3. Therefore, the first groove 111 and the second groove 112, which extend along the first direction, allow the user's wrist horizontally positioning along the first direction d1.

The second adjusting arm 123 connects to the sensing module 130 along the second direction d2, and the first direction d1 is perpendicular to the second direction d2. Therefore, by using the second adjusting arm 123, the sensing module 130 can be moved to a position corresponding to the first groove 111 or a position corresponding to the second groove 112. Namely, the second adjusting arm 123 can adjust the position of the sensing module 130 along the second direction d2, such that the sensing module 130 can be moved across the first groove 111 and the second groove 112.

The first adjusting arm 122 connects the base 121 and the second adjusting arm 123 along the third direction d3. The first adjusting arm 122 is utilized to adjust and decide the distance between the first carrier 110 and the sensing module 130. When the user's wrist is rested on the first carrier 110, the first adjusting arm 122 can adjust the position of the sensing module 130, such that the sensing module 130 can align with the wrist.

The sensing module 130 includes a second carrier 131 and a pressure sensor 132. The second carrier 131 is connected to the second adjusting arm 123, and the pressure sensor 132 is disposed on the second carrier 131. The pressure sensor 132 is disposed facing towards the first groove 111 and the second groove 112, and the second carrier 131 is disposed between the second adjusting arm 123 and the pressure sensor 132.

Therefore, the second adjusting arm 123 can adjust the position of the pressure sensor 132 along the second direction d2, such that the pressure sensor 132 is able to facing towards the first groove 111 or the second groove 112. The first adjusting arm 122 can move the pressure sensor 132 along the third direction d3, to adjust the distance between the pressure sensor 132 and the first carrier 110. As a result, the pressure sensor 132 is moved towards the first groove 111 or the second groove 112.

For example, in some embodiments of the invention, a biomedical signal sensing method for taking a biomedical signal from a user includes following steps. The biomedical signal sensing device 100 is provided, and a wrist of the user is rested on the first carrier 110 of the biomedical signal sensing device 100, and a position of the wrist relative to the first carrier 110 on the first direction d1 is determined by the first carrier 110. Namely, a first lateral wrist print of the wrist that is close to a palm cam be aligned with a component of the first carrier 110, to determine the position of the wrist on the first direction d1.

Then, the biomedical signal sensing method further adjusting the position of the sensing module 130 on the second direction d2 by using the frame 120. For example, in this embodiment, the second adjusting arm 123 of the frame 120 moves the sensing module 130 to the positions that substantially faces towards a portion of the first groove 111.

After the position of the sensing module 130 on the second direction d2 is determined, the first adjusting arm 122 of the frame 120 adjusts the position of the sensing module 130 on the third direction d3 and forces the sensing module 130 touching the wrist.

Then, the first carrier 110 can read the biomedical signal by the sensing module 130. More particularly, the pressure sensor 132 of the sensing module 130 is configured to measure pulse signals while touching user's wrist, and the information of the pulse signals is sent and stored in a processor (not shown in FIG. 1) which is electrically connected to the sensing module 130. Additionally, the frame 120 can also electrically connected to the processor, such that the first adjusting arm 122 and the second adjusting arm 123 of the frame 120 can be controlled by the processor.

Therefore, the first carrier 110 of the biomedical signal sensing device 100 is adjusted to secure the wrist, and the wrist can align the edge of the first carrier 110. The second adjusting arm 123 of the biomedical signal sensing device 100 is configured to move the sensing module 130 to the position corresponding to the first groove 111 or the second groove 112 where the wrist is rested on. The first adjusting arm 122 is configured to move the sensing module 130 to touch the wrist. The biomedical signal sensing device 100 and the biomedical signal sensing method provided above allow the user measuring biomedical signal precisely.

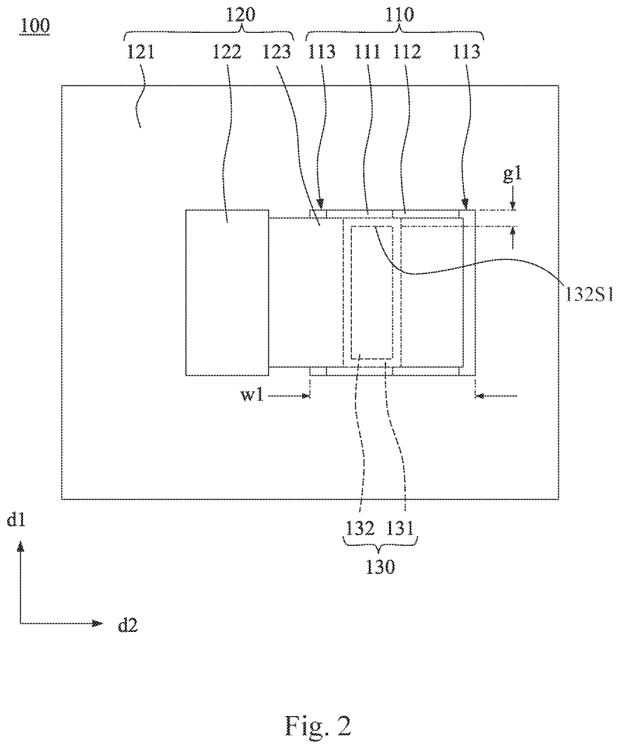

FIG. 2 is a schematic top view of the biomedical signal sensing device 100, according to some embodiments of the invention, in which the pressure sensor 132 shielded by the second adjusting arm 123 is illustrated in dashed lines. Referring to FIG. 2, along the first direction d1, a minimum distance g1 between the pressure sensor 132 and the first carrier 110 is not greater than 20 mm. More particularly, the minimum distance g1 between the pressure sensor 132 and the first carrier 110 ranges from 1 mm to 20 mm.

More particularly, the first carrier 110 has an edge 113. Along the first direction d1, a minimum distance g1 between the sensing module 130 and the edge 113 of the first carrier 110 ranges from 1 mm to 20 mm. For example, the edge 113 of the first carrier 110 is the edge that is adjacent the palm. The pressure sensor 132 also has an edge 132S1 that is adjacent the palm. The minimum distance between the edge 113 of the first carrier 110 and the edge 132S1 of the pressure sensor 132 ranges from 1 mm to 20 mm.

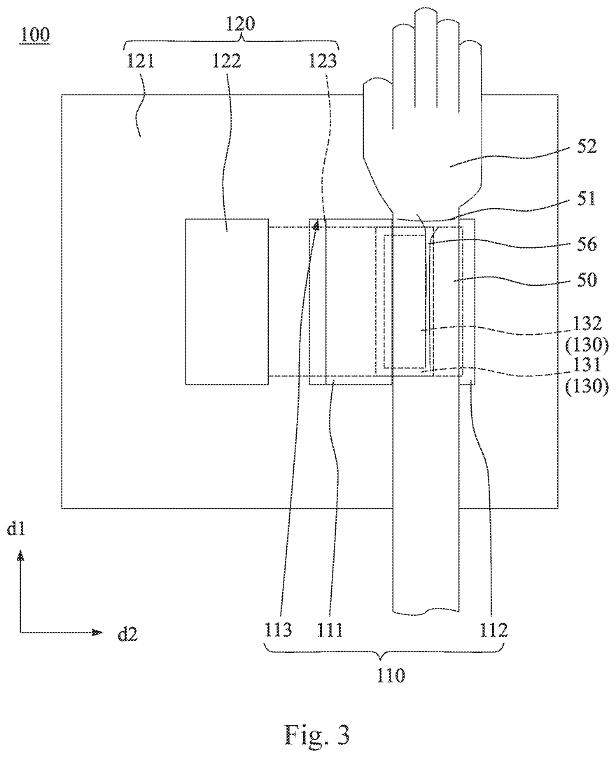

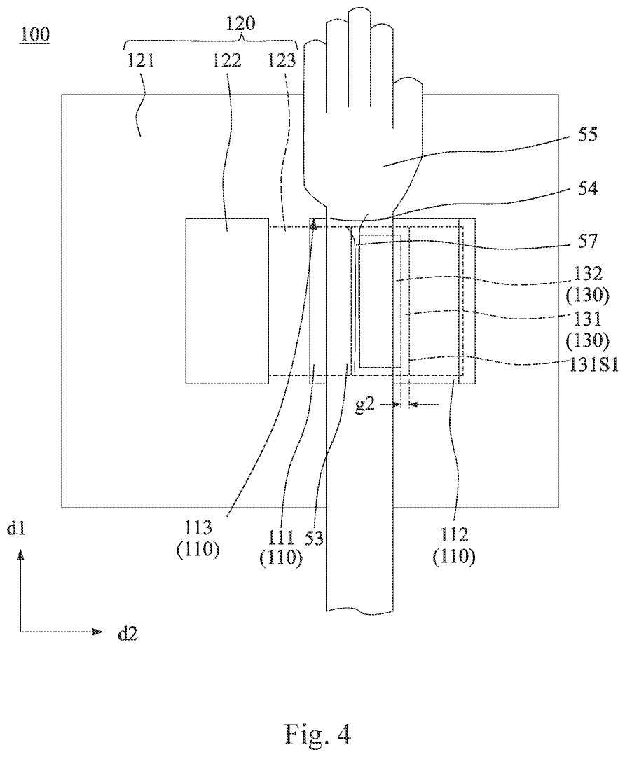

FIG. 3 and FIG. 4 are schematic top views of the biomedical signal sensing device 100 at different operation states, according to some embodiments of the invention, in which the second adjusting arm 123 and the pressure sensor 132 are illustrated in dashed lines. FIG. 3 illustrates that the user's wrist is rested in the first groove 111, and FIG. 4 illustrates that the user's wrist is rested in the second groove 112. For example, referring to FIG. 2 to FIG. 4, in some embodiments, the position of the pressure sensor 132 is close to the edge 113 of the first carrier 110 on the first direction d1. The first lateral wrist print 51 of the wrist 50, which is adjacent the wrist 50, can align with the edge 113 of the first carrier 110 when the wrist 50 is rested in the second groove 112. Therefore, the cun of the wrist 50 is located within a reachable range of the pressure sensor 132. Hereafter, the first lateral wrist print 51 is the lateral wrist print closet to the palm among the visible lateral wrist prints when the user's palm faces upwards.

When another wrist 53 of the user is rested in the first groove 111, the first lateral wrist print 54 of the wrist 53, which is adjacent the palm 55, can align with the edge 113 of the first carrier 110, such that the cun of the wrist 53 is located within the reachable range of the pressure sensor 132.

Namely, according to the biomedical signal sensing method of this embodiment, the step of determining the position of the wrist 50 relative to the first carrier 110 on the first direction d1 includes aligning the first lateral wrist print 51 of the wrist 50 adjacent the palm 52 with the edge 113 of the first carrier 110. Thus the position of the wrist 50 is determined, and the cun of the wrist 50 is well positioned and is easily detected by the sensing module 130.

On the other hand, the step of determining the position of the other wrist 53 relative to the first carrier 110 on the first direction d1 includes aligning the first lateral wrist print 54 of the wrist 53 adjacent the palm 55 with the edge 113 of the first carrier 110. Thus the position of the wrist 53 is determined, and the cun of the wrist 53 is well positioned and is easily detected by the sensing module 130.

More particularly, the wrist 50 is user's left wrist and faces upwards when the wrist 50 is rested in the second groove 112. The wrist 53 is user's right wrist and faces upwards when the wrist 53 is rested in the first groove 111.

Additionally, according to the biomedical signal sensing method of this embodiment, the step of adjusting the position of the sensing module 130 on the second direction d2 by using the frame 120 includes aligning an edge of the tendon of flexor carpi radialis 56 of the wrist 50 or an edge of the tendon of flexor carpi radialis 57 of the wrist 53 with an edge of the second carrier 131 of the sensing module 130. Thus the sensing module 130 is positioned above the cun of the wrist 50 or wrist 53 and is able to detect the biomedical signal of the wrist 50 or wrist 53.

More particularly, as illustrated in FIG. 4, in some embodiments, a minimum distance g2 between the pressure sensor 132 and an edge 131S1 is not greater than 8 mm, along the second direction d2. For example, minimum distance g2 ranges from 0 mm to 20 mm, in some embodiments of the invention, but the invention is not limited to.

In this embodiment, a width w1 of the first carrier 110 on the second direction d2 ranges from 90 mm to 160 mm thereby having sufficient space for arranging the first groove 111 and the second groove 112.

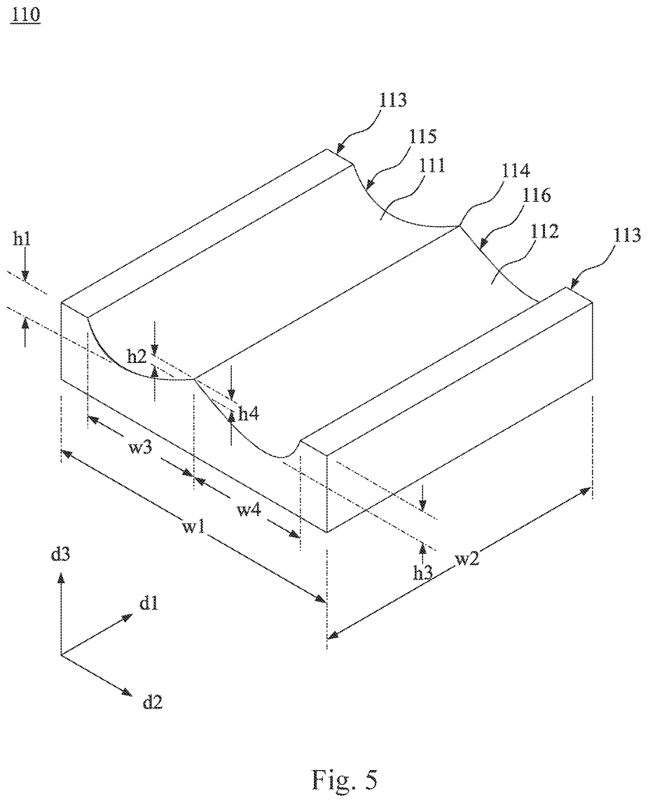

FIG. 5 is an oblique view of the first carrier 110, according to some embodiments of invention. Referring to FIG. 5, a width w2 of the first carrier 110 on the first direction d1 ranges from 40 mm to 80 mm thereby having sufficient space for supporting the wrist 50 (as shown in FIG. 3) or the wrist 53 (as shown in FIG. 4).

More particularly, in this embodiment, the first groove 111 of the first carrier 110 has a width w3 on the second direction d2, and the width w3 ranges from 15 mm to 60 mm, thereby having sufficient space for supporting the wrist 53 (as shown in FIG. 4). For example, the first groove 111 is designed for supporting the right wrist. The second groove 112 of the first carrier 110 has a width w4 on the second direction d2, and the width w4 ranges from 15 mm to 60 mm, thereby having sufficient space for supporting the wrist 50 (as shown in FIG. 3). For example, the second groove 112 is designed for supporting the left wrist.

On the other hand, a spacer ridge 114 is formed between the first groove 111 and the second groove 112. The first groove 111 has a first depth h1 at a side away from the spacer ridge 114. The first groove 111 has a second depth h2 at a side adjacent the spacer ridge 114. The first depth h1 is greater than the second depth h2. The second groove 112 has a third depth h3 at a side away from the spacer ridge 114. The second groove 112 has a fourth depth h4 at a side adjacent the spacer ridge 114. The third depth h3 is greater than the fourth depth h4. More particularly, in this embodiment, the maximum depth h1 of the first groove 111 ranges from 5 mm to 30 mm, and the maximum depth h3 of the second groove 112 ranges from 5 mm to 45 mm. Therefore, when the wrist 50 (as shown in FIG. 3) is rested on the second groove 112, the cun of the wrist 50 faces upwards for being detected. When the wrist 53 (as shown in FIG. 4) is rested on the first groove 111, the cun of the wrist 53 faces upwards for being detected.

In this embodiment, the first groove 111 has a first position end 115 at a terminal along the first direction, and the second groove 112 has a second position end 116 at a terminal along the first direction d1. The first position end 115 and the second position end 116 are arranged along the second direction d2. Thus the first position end 115 of the first groove 111 is designed for positioning the wrist 53, and the second position end 116 of the second groove 112 is designed for positioning the wrist 50.

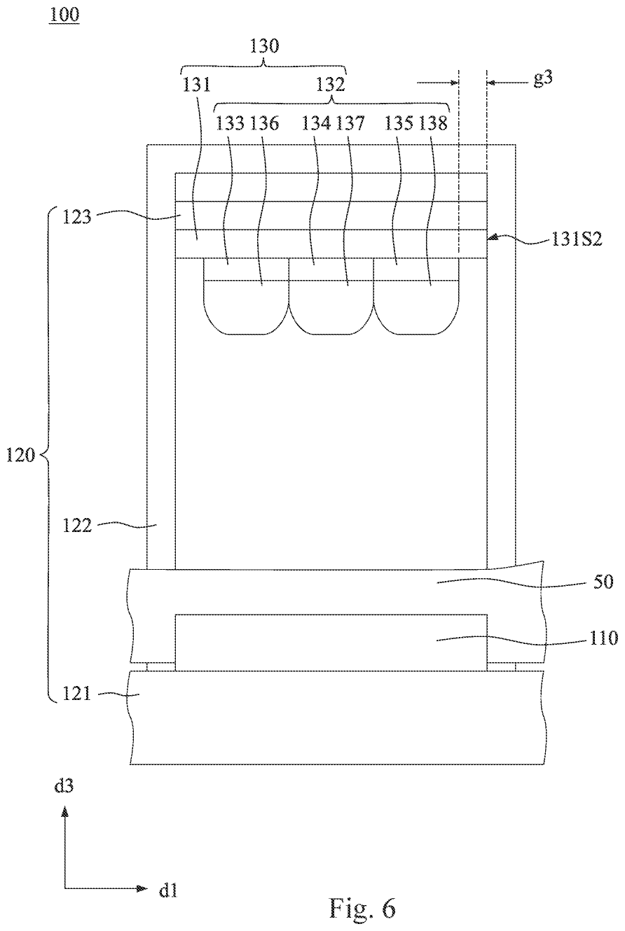

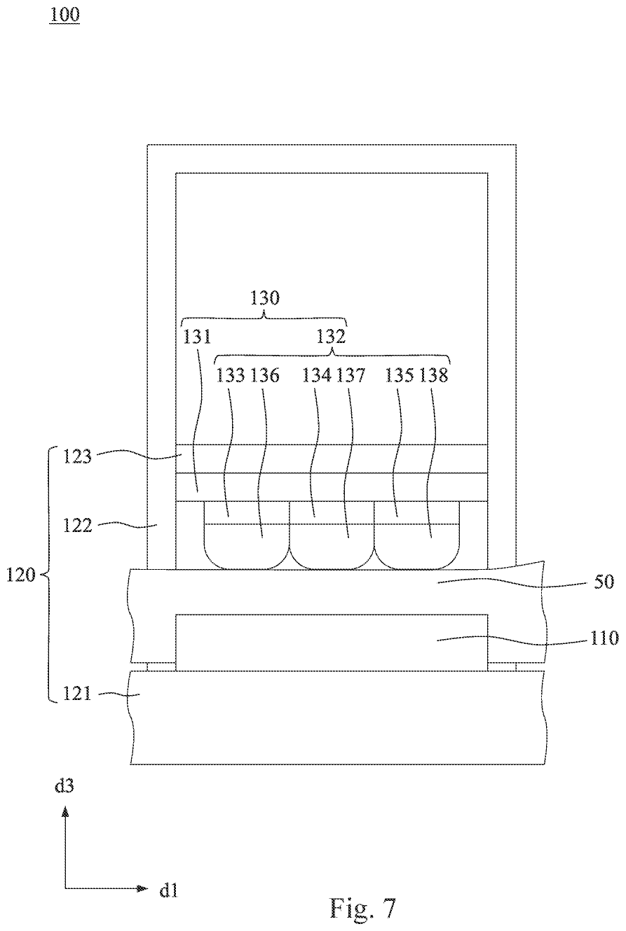

FIG. 6 and FIG. 7 are schematic side views of the biomedical signal sensing device 100 at different operation stages, according to some embodiments of the invention, in which FIG. 6 is the stage before the position of the sensing module 130 is adjusted along the third direction d3, and FIG. 7 is the stage after the position of the sensing module 130 is adjusted along the third direction d3.

Please referring to FIG. 6, in this embodiment, a minimum distance g3 between the pressure sensor 132 and the edge 131S2 of the second carrier 131 on the first direction d1 is not greater than 20 mm. For example, the minimum distance g3 ranges from 1 mm to 20 mm in this embodiment. Thus the sensing module 130 is able to detect the biomedical signal of the cun of the wrist 50.

More particularly, the pressure sensor 132 includes a first pressure sensing unit 133, a first air bag 136, a second pressure sensing unit 134, a second air bag 137, a third pressure sensing unit 135, and a third air bag 138. The first air bag 136 is disposed on the first pressure sensing unit 133, and the first pressure sensing unit 133 is disposed between the second carrier 131 and the first air bag 136. The second air bag 137 is disposed on the second pressure sensing unit 134, and the second pressure sensing unit 134 is disposed between the second carrier 131 and the second air bag 137. The third air bag 138 is disposed on the third pressure sensing unit 135, and the third pressure sensing unit 135 is disposed between the second carrier 131 and the third air bag 138.

The first pressure sensing unit 133, the second pressure sensing unit 134, and the third pressure sensing unit 135 are arranged along the first direction d1, and thus the first air bag 136, the second air bag 137, and the third air bag 138 are also arranged along the first direction d1. Therefore, when the pressure sensor 132 is disposed corresponding to the cun of the wrist 50, the first air bag 136, the second air bag 137, and the third air bag 138 are positioned relative to chi, guan, cun of the wrist 50.

Furthermore, in this embodiment, before the pressure sensor 132 touching the wrist 50, the air pressures of the first air bag 136, the second air bag 137, and the third air bag 138 can be the same or different. Therefore, the third air bag 138, the second air bag 137, and the first air bag 136 can detect the biomedical signals from cun, guan, chi, respectively. The biomedical signals can be, for example pulse signals.

For example, the air pressures of the first air bag 136, the second air bag 137, and the third air bag 138 range from 80 mmHg to 155 mmHg.

In the biomedical signal sensing method of this embodiment, the step of adjusting the position of the sensing module 130 on the third direction d3 by using the frame 120 includes obtaining a pressure value from the pressure sensor 132 of the sensing module 130. Then the position of the sensing module 130 on the third direction is decided when the pressure value is raised from an initial pressure value to a target pressure value.

Namely, before the sensing module 130 touches the wrist 50 on the first carrier 110, the air pressures of the first air bag 136, the second air bag 137, and the third air bag 138 are initial pressure values, and the initial pressure values ranges from 80 mmHg to 110 mmHg.

Please refer to FIG. 7. When the sensing module 130 touches the wrist 50, the first air bag 136, the second air bag 137, and the third air bag 138 are deformed and the pressure values obtained by the pressure sensor 132 is increased. The process of adjusting the sensing module 130 by the frame 120 stops when the pressure values obtained by the pressure sensor 132 reach the target pressure value. For example, the target pressure value ranges from frame 120 mmHg to 155 mmHg.

In this embodiment, the different between the initial pressure value and the target pressure value is in a range from 5 mmHg to 40 mmHg. The sensing module 130 can successfully obtain the biomedical signals by touching the wrist 50.

For example, the first pressure sensing unit 133, the second pressure sensing unit 134, and the third pressure sensing unit 135 can be piezoelectric sensors, piezoresistive sensors, capacitive sensor, or other suitable flexible pressure sensors, and the invention is not limited to.

According to some embodiments of the invention, the biomedical signal sensing device and the biomedical signal sensing method can align the sensing module with the first carrier by using the frame. Thus the biomedical signal sensing device can precisely detect the biomedical signal when user rests the wrist on the first carrier.

Although the present invention has been described in considerable detail with reference to certain embodiments thereof, other embodiments are possible. Therefore, the spirit and scope of the appended claims should not be limited to the description of the embodiments contained herein.

It will be apparent to those skilled in the art that various modifications and variations can be made to the structure of the present invention without departing from the scope or spirit of the invention. In view of the foregoing, it is intended that the present invention cover modifications and variations of this invention provided they fall within the scope of the following claims and their equivalents.

What is claimed is:

1. A biomedical signal sensing device, comprising:
a frame, comprising:
a base;
a first adjusting arm; and
a second adjusting arm connecting to the base through the first adjusting arm;
a first carrier disposed on the base, the first carrier comprising:
a first groove; and
a second groove disposed adjacent the first groove; and
a sensing module connected to the first adjusting arm through the second adjusting arm, the sensing module comprising:
a second carrier connected to the second adjusting arm; and
a pressure sensor disposed on the second carrier, wherein the pressure sensor faces towards the first groove or the second groove, the first groove and the second groove extend along a first direction, the second adjusting arm extends along a second direction, the first adjusting arm extends along a third direction, and the first direction, the second direction, and the third direction are perpendicular to each other.

2. The biomedical signal sensing device of claim 1, wherein a minimum distance between the sensing module and an edge of the first carrier ranges from 1 mm to 20 mm.

3. The biomedical signal sensing device of claim 1, wherein a width of the first carrier along the second direction ranges from 90 mm to 160 mm.

4. The biomedical signal sensing device of claim 1, wherein a minimum distance between the sensing module and an edge of the second carrier ranges from 0 mm to 20 mm, along the second direction.

5. The biomedical signal sensing device of claim 1, wherein the pressure sensor comprises:
a first pressure sensing unit;
a first air bag disposed on the first pressure sensing unit;
a second pressure sensing unit;
a second air bag disposed on the second pressure sensing unit;
a third pressure sensing unit; and
a third air bag disposed on the third pressure sensing unit, wherein the first pressure sensing unit, the second pressure sensing unit, and the third pressure sensing unit are arranged along the first direction.

6. The biomedical signal sensing device of claim 5, wherein the first air bag, the second air bag, and the third air bag have different sizes.

7. The biomedical signal sensing device of claim 5, wherein the first air bag, the second air bag, and the third air bag have different air pressures.

8. The biomedical signal sensing device of claim 5, wherein air pressures of the first air bag, the second air bag, and the third air bag range from 80 mmHg to 155 mmHg.

9. The biomedical signal sensing device of claim 1, further comprising a spacer ridge between the first groove and the second groove,
wherein a first depth is defined at a side of the first groove away from the spacer ridge, a second depth is defined at a side of the first groove near the spacer ridge, the first depth is larger than the second depth; and
wherein a third depth is defined at a side of the second groove away from the spacer ridge, a fourth depth is defined at a side of the second groove near the spacer ridge, the third depth is larger than the fourth depth.

10. The biomedical signal sensing device of claim 1, wherein the first groove has a first position end at a terminal along the first direction, the second groove has a second position end at a terminal along the first direction, and the first position end and the second position end are arranged along the second direction.

11. The biomedical signal sensing device of claim 1, wherein the first groove is configured to rest a right wrist, and the second groove is configured to rest a left wrist.

12. A biomedical signal sensing method, for taking a biomedical signal from a user, the method comprising:
providing a biomedical signal sensing device, which comprises a frame, a first carrier, and a sensing module;
resting a wrist of the user on the first carrier of the biomedical signal sensing device, and determining a position of the wrist relative to the first carrier on a first direction;
adjusting a position of the sensing module on a second direction by the frame;
adjusting the position of the sensing module on a third direction by the frame and forcing the sensing module touching the wrist; and
reading the biomedical signal by the sensing module.

13. The biomedical signal sensing method of claim 12, wherein determining a position of the wrist relative to the first carrier on a first direction comprises:
aligning an edge of the first carrier to a first lateral wrist print of the wrist that is close to a palm.

14. The biomedical signal sensing method of claim 12, wherein adjusting a position of the sensing module on a second direction by the frame comprises:
aligning an edge of a second carrier of the sensing module to a tendon of flexor carpi radialis of the wrist, such that the sensing module is above a can of the wrist.

15. The biomedical signal sensing method of claim 12, wherein adjusting the position of the sensing module on a third direction by the frame comprises:
obtaining a pressure value from a pressure sensor of the sensing module; and
deciding the position of the sensing module on the third direction when the pressure value raises from an initial pressure value to a target pressure value.

16. The biomedical signal sensing method of claim 15, wherein the initial pressure value ranges from 80 mmHg to 110 mmHg, and the target pressure value ranges from frame 120 mmHg to 155 mmHg.

17. The biomedical signal sensing method of claim 15, wherein obtaining a pressure value from a pressure sensor of the sensing module comprises:
obtaining pressure values of a plurality of air bags of the pressure sensor.

18. The biomedical signal sensing method of claim 17, wherein initial pressure values of the air bags are different.

19. The biomedical signal sensing method of claim 15, wherein a difference between the initial pressure value and the target pressure value ranges from 5 mmHg to 40 mmHg.

20. The biomedical signal sensing method of claim 15, wherein adjusting a position of the sensing module on a second direction by the frame comprises:
adjusting the frame such that a minimum distance between a pressure sensor of the sensing module and an edge of the first carrier is ranged from 1 mm to 20 mm.
